(19) United States
(12) Patent Application Publication
(10) Pub. No.: US 2008/0318880 A1
(43) Pub. Date: Dec. 25, 2008

(54) Neoadjuvant treatment of Breast Cancer

(75) Inventor: Lorne J. Brandes, Winnipeg (CA)

Correspondence Address: ROTHWELL, FIGG, ERNST & MANBECK, P.C., 1425 K STREET, N.W., SUITE 800, WASHINGTON, DC, 20005, US

(73) Assignee: The University of Manitoba, Winnipeg (CA)

(21) Appl. No.: 12/198,320

(22) Filed: Aug. 26, 2008

Related U.S. Application Data
(63) Continuation of application No. 10/526,535, filed on Aug. 26, 2005, now abandoned.
(60) Provisional application No. 60/407,242, filed on Sep. 3, 2002.

(51) Int. Cl. A61K 31/704; A61K 31/195; A61K 31/337

(52) U.S. Cl. 514/34; 514/651; 514/449

(57) ABSTRACT

Neoadjuvant treatment of inflammatory or T3 to T4 breast cancer is carried out by administering to such patients a number of cycles of chemotherapy treatment in which DPPE first is administered followed by a combination of anthracyclines and taxanes.

### FIELD OF THE INVENTION

The present invention relates to the treatment of breast cancer.

### BACKGROUND OF THE INVENTION

One of the major chemotherapeutic treatments is that of malignant growth (cancer) in humans. The objective of chemotherapy is the total extermination of clonogenic tumor or malignant cells, with minimal damage to the patient. However, one of the major limitations of the chemotherapeutic approach for managing human cancer is the general inability of anticancer drugs to discriminate between normal and tumorous cells. Anti-neoplastic agents have the lowest therapeutic indicies of any class of drugs used in humans and hence produce significant and potentially life-threatening toxicities. Certain commonly-used anti-neoplastic agents have unique and acute toxicities for specific tissues. For example, the vinca alkaloids possess significant toxicity for nervous tissues, while adriamycin has specific toxicity for heart tissue and bleomycin has for lung tissue. In general, almost all members of the major categories of anti-neoplastic agents have considerable toxicities for normal cells of gastrointestinal, epidermal and myelopoietic tissues.

Generally, the dose-limiting consideration for chemical management of cancer in humans is the toxicity that anti-neoplastic agents have for the pluripotent stem cells of myelopoietic tissue. This toxicity arises from the fact that most anticancer drugs function preferentially against proliferating cells but with no significant capacity to discriminate between cycling normal and cycling tumor tissues.

In certain types of locally-advanced breast cancer, specifically inflammatory or T3 to T4 breast cancer, there is applied a treatment with chemotherapeutic agents prior to surgical removal of the tumor, in order to reduce the size of tumor. T3 tumors are tumors sized >3 and <4 cm. T3 tumors may be operable or inoperable depending on where in the breast they are located. For example, they are often inoperable if close to the chest wall, especially in small breasts. T4 tumors are tumors sized >4 cm and generally are inoperable. Inflammatory breast cancer infiltrates the lymphatics of the skin, is usually a diffuse tumor and very high grade in term of malignancy.

In U.S. Pat. Nos. 6,288,799, 5,859,065, 5,708,329, 5,747,543 and 5,618,846, all assigned to University of Manitoba and the disclosures of which are incorporated herein by reference, there is described an improved method for the in vivo chemotherapeutic treatment of cancer in which there is first administered a compound which inhibits normal cell proliferation while promoting malignant cell proliferation, specifically a potent antagonist selective for intracellular histamine receptors, in an amount sufficient to inhibit the binding of intracellular histamine to the receptors in normal and malignant cells. Following sufficient time to permit the inhibition of binding of intracellular histamine, a chemotherapeutic agent is administered. An enhanced toxic effect on the cancer cells from the chemotherapeutic agent is obtained while any adverse effect of the chemotherapeutic agent on normal cells, particularly bone marrow and gastro-intestinal cells, is significantly ameliorated. One useful compound which inhibits normal cell proliferation while promoting malignant cell proliferation is N,N-diethyl-2-[4-(phenylmethyl)-phenoxy]ethanamine, abbreviated herein as DPPE.

### SUMMARY OF INVENTION

It has now surprisingly been found, in a Phase II clinical trial, the procedure described in the aforementioned patents when using a combination of an anthracycline chemotherapeutic agent and a taxane therapeutic agent is an effective procedure in neoadjuvant treatment of inflammatory breast cancer or T3 to T4 breast cancer. In addition, the procedure leads to long term survival post surgery.

Accordingly, in one aspect, the present invention provides a method of neoadjuvant chemotherapy in patients with inflammatory or T3 to T4 breast cancer, which comprises administering to said patients a plurality of cycles of chemotherapy at predetermined intervals until cancerous tissue is reduced to an operable size or is in remission, wherein each said cycle comprises:

(a) first administering to said patients at least one diphenyl compound of the formula:

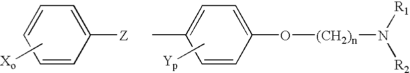

wherein X and Y are each fluorine, chlorine or bromine, Z is an alkylene group of 1 to 3 carbon atoms or ═C═O, or the phenyl groups are joined to form a tricyclic ring, o and p are 0 or 1, R₁ and R₂ are each an alkyl group containing 1 to 3 carbon atoms or are joined together to form a heterocyclic ring with the nitrogen atom and n is 1, 2 or 3, or pharmaceutically-acceptable salts thereof, and

(b) following sufficient time to permit inhibition of binding of intracellular histamine, subsequently administering to the patient an anthracycline chemotherapeutic agent and a taxane chemotherapeutic agent.

In the application of the present invention, the diphenyl compound and the chemotherapeutic agents are generally administered by intravenous infusion. In one preferred procedure, a solution of the diphenyl compound is administered to the patient over a desired period of time prior to administration of the chemotherapeutic agents and a solution of the chemotherapeutic agents in combination with the diphenyl compound then is administered for the period of administration of the chemotherapeutic agents. If desired, a solution of the diphenyl compound is administered after completion of the administration of the chemotherapeutic agents for a desired period of time to ameliorate side effects from the administration of the chemotherapeutic agents.

### GENERAL DESCRIPTION OF INVENTION

In the present invention, a diphenyl compound is used which is a potent antagonist of histamine binding at the intracellular histamine receptor and is administered in an amount sufficient to inhibit the binding of intracellular histamine at the intracellular binding site (H_{IC}) in normal cells. Such compounds exhibit a pKi of at least about 5, preferably at least about 5.5.

Specific potent compounds which are useful in the present invention are diphenyl compounds of the formula:

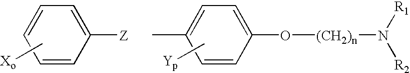

wherein X and Y are each fluorine, chlorine or bromine, Z is an alkylene group of 1 to 3 carbon atoms or ═C═O, o and p are 0 or 1, R₁ and R₂ are each alkyl groups containing 1 to 3 carbon atoms or are joined together to form a hetero-ring with the nitrogen atom and n is 1, 2 or 3. Pharmaceutically-acceptable salts of the diphenyl compounds may be employed.

Alternatively, the benzene rings may be joined to form a tricyclic ring, in accordance with the structure:

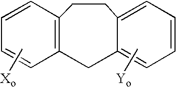

In one preferred embodiment, the group

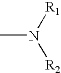

is a diethylamino group, although other alkylamino groups may be employed, such as dimethylamino, and, in another preferred embodiment, a morpholino group, although other heterocyclic ring groups may be employed, such as piperazino. o and p are usually 0 when Z is an alkylene group and n may be 2. In one particularly preferred embodiment, Z is —CH₂—, n is 2, o and p are each 0 and

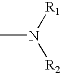

is a diethylamino group. This compound, namely N,N-diethyl-2-[4-(phenylmethyl)-phenoxy]ethanamine, which may be in the form of the free base or in the form of its hydrochloride or other pharmaceutically-acceptable salt, is abbreviated herein as DPPE. In addition to a methylene group linking the benzene rings, other linking groups may be employed, such as ═C═O. Other substitutents may be provided on the benzene rings in addition to the halogen atoms, for example, an imidazole group.

The diphenyl compound employed in the present invention is administered to the patient in any convenient manner, such as by intravenous injection of a solution thereof in an aqueous pharmaceutically-acceptable vehicle. The diphenyl compound is administered to the patient over a period of time before administration of the chemotherapeutic agents.

The chemotherapeutic agents employed herein are anthracyclines, preferably doxorubicin and epirubicin; and taxanes, preferably Taxol (Trademark of Bristol-Myers Squibb for paclitaxel) and Taxotere (Trademark of Aventis Pharma for docetaxel). The mixture of chemotherapeutic agents is administered in any manner consistent with their normal manner of administration in conventional breast cancer therapy, usually by intravenous infusion of a solution thereof.

The administration of the diphenyl compound to the patient prior to administration of the chemotherapeutic agents is necessary in order to permit the diphenyl compound to inhibit the binding of intracellular histamine in normal and malignant cells and thereby, in effect, shut down the proliferation of the normal cells, but increase proliferation of malignant cells.

The length of time prior to administration of the chemotherapeutic agents that the diphenyl compound is administered depends on the diphenyl compound, its mode of administration and the size of the patient. Generally, the diphenyl compound is administered to the patient for about 30 to about 90 minutes, preferably about 60 minutes, prior to administration of the chemotherapeutic agents.

The quantity of diphenyl compound administered to the patient depends on the side effects to be ameliorated, but should be at least sufficient to inhibit binding of intracellular histamine in normal cells. The quantity required to achieve the beneficial effects of the present invention depends upon the diphenyl compound employed, the chemotherapeutic agents employed and the quantity of such agents employed.

In general, the quantity of diphenyl compound employed in humans is from about 8 to about 320 mg/M² of human to which the diphenyl compound is administered, with about 8 and 240 mg/M² being the optimal dose for gastro-intestinal and bone marrow protection, respectively. Over this dose range, the present invention is able to achieve an enhanced chemotherapeutic effect on breast cancer cells while, at the same time, also protecting normal cells from damage by the chemotherapeutic agents in a wide variety of circumstances where traditional chemotherapy leads to damage of normal cells or tissues not involved in the disease process.

In the neoadjuvant treatment of inflammatory or T3 to T4 breast cancer, the diphenyl compound preferably is used in an amount of about 3 to about 10 mg/kg of patient, administered intravenously over a period of about 30 to about 90 minutes prior to administration of the chemotherapeutic agents and continuing for the period of administration of the chemotherapy agent. In the specific Phase II clinical trial described herein, there was employed 6 mg/kg of DPPE in the form of its hydrochloride salt, administered intravenously as an aqueous solution thereof over 80 minutes, with the last twenty minutes being accompanied by infusion of the chemotherapeutic agents, followed by the intravenous administration of an aqueous solution at a dose of 2.5 mg/kg of DPPE for 180 minutes accompanied by the infusion of Taxol® or for 60 minutes accompanied by the infusion of Taxotere®.

A second regimen for DPPE/Taxotere® treatment is the intravenous administration of an aqueous solution of DPPE for 80 minutes, with the last 20 minutes being accompanied by infusion of the Taxotere®, followed by infusion of Taxotere® alone for 40 minutes.

The chemotherapy agents which are employed herein preferably are used in a total amount of 75 to about 225 mg/M² of patient consistent with the identity of the chemotherapy agent. The chemotherapeutic agents may be administered in an amount of about 50 to about 60 mg/M² of patient for doxorubicin or epirubicin, about 175 to about 225 mg/M² of Taxol® and about 75 to about 100 mg/M² of Taxotere®. In the specific Phase II clinical trial described herein, there was employed 50 mg/M² of doxorubicin or epirubicin, and 175 mg/M² of Taxol® or 75 mg/M² of Taxotere®, administered over the last 20 minutes of infusion of the DPPE solution and over a further 180 minutes for Taxol® or 60 minutes for Taxotere®, accompanied by infusion of a 2.5 mg/kg of DPPE solution.

As noted above, patients with inflammatory breast cancer or T3 to T4 breast cancer are subjected to a number of cycles of chemotherapy at predetermined intervals to reduce the size of the tumor to an operable size. The number of cycles for each patient is generally about 5 to about 8 cycles, with about 21 to about 28 days between each cycle. In the Phase II clinical trial, patients were subjected to 6 cycles at time intervals of 21 days.

As set forth herein, a Phase II clinical trial was conducted on patients having inflammatory or T3 to T4 breast cancer in which patients were administered DPPE followed by doxorubicin or epirubicin and Taxol® or Taxotere®. Various data from the clinical trial were collected and analyzed.

The results of this trial showed that DPPE along with doxorubicin/epirubicin and Taxol®/Taxotere® was an effective neoadjuvant treatment which lead to long term survival post surgery.

### EXAMPLE

This Example illustrates the neoadjuvant treatment of inflammatory or T3-T4 breast cancer.

A Phase II clinical trial was carried out in which patients (N=8) with inflammatory (N=7) and T3 to T4 (N=1) breast cancer were treated with a combination of DPPE and epirubicin (EPI)/Taxol® (N=5), a combination of DPPE and doxorubicin (DOX)/Taxol® (N=2) and DPPE and a combination of DPPE and epirubicin/Taxotere® (N=1). DPPE was administered at a dose of 6 mg/M² over 80 minutes with a combination of epirubicin or doxorubicin at a dose of 50 mg/M² and Taxol® at a dose of 175 mg/M² or Taxotere® at a dose of 75 mg/M² over the last 20 minutes and during a further 180 minutes for Taxol® or 60 minutes for Taxotere®, at a dose of 2.5 mg/kg. The treatment was repeated at 21 day intervals for 6 cycles. The eight patients with inflammatory or T3 to T4 breast cancer had no previous chemo- or radiotherapy. When the chemotherapy cycles were complete, the cancerous tissue was removed and the patients observed.

The results obtained are shown in Table I. In this Table, the abbreviation TTP stands for time to progression and the abbreviation OS stands for overall survival. Two long-term survivors (45+ and 53+ months) had mixed high-grade tumors pre-treatment but no high-grade component post-surgery. A third patient with only high-grade cells or cytology, had a clinical/pathological complete remission. This patient remains disease-free at 55+ months.

These findings are compatible with the hypothesis advanced in copending U.S. Applications Nos. 60/331,242 filed Nov. 9, 2001 and 10/494,961 filed Jan. 3, 2005, the disclosures of which are incorporated herein by reference (WO03/039526), in the names of Lorne Brandes and Mark Vincent, that DPPE-based chemotherapy may preferentially target high-grade malignant cell populations.

### SUMMARY OF INVENTION

In summary of this disclosure, the present invention provides a neoadjuvant chemotherapeutic treatment of inflammatory or T3 to T4 breast cancer. Modifications are possible within the scope of the invention.

TABLE I
SUMMARY OF RESULTS
NEOADJUVANT THERAPY

| No. | Cycles | Response* | | | TTP | OS |
|---|---|---|---|---|---|---|
| Patients | (Total) | CR | PR | NR | (Median, mos.) | |
| 8† | 46 | 4/2* | 3/4* | 1/2* | 21 | 43+ |

†5 DPPE/EPI/TAXOL; 2 DPPE/DOX/TAXOL; 1 DPPE/EPI/TAXOTERE
*Pathological criteria

What I claim is:

1. A method of neoadjuvant chemotherapy in patients with inflammatory breast cancer or T3 or T4 breast cancer which comprises administering to said patient a plurality of cycles of chemotherapy at predetermined intervals until cancerous tissue is reduced to an operable size or is in remission, wherein each said cycle comprises:
(a) first administering to said patients at least one diphenyl compound of the formula: 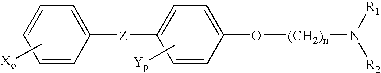
wherein X and Y are each fluorine, chlorine or bromine, Z is an alkylene group of 1 to 3 carbon atoms or ═C═O, or the phenyl groups are joined to form a tricyclic ring, o and p are 0 or 1, R₁ and R₂ are each an alkyl group containing 1 to 3 carbon atoms or are joined together to form a heterocyclic ring with the nitrogen atom and n is 1, 2 or 3, or pharmaceutically-acceptable salts thereof, and
(b) following sufficient time to permit inhibition of binding of intracellular histamine, subsequently administering to the patient an anthracycline chemotherapeutic agent and a taxane chemotherapeutic agent.

2. The method of claim 1 wherein the group 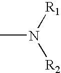
is a diethylamino group, a dimethylamino group, a morpholino group or a piperazino group.

3. The method of claim 1 wherein the group 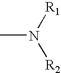
is a diethylamino group, Z is —CH₂, n is 2 and o and p are each 0.

4. The method of claim 3 wherein diphenyl compound is in the form of a hydrochloride salt.

5. The method of claim 1 wherein said anthracycline chemotherapeutic agent is doxorubicin or epirubicin.

6. The method of claim 4 wherein said anthracycline chemotherapeutic agent is doxorubicin or epirubicin.

7. The method of claim 1 wherein said taxane chemotherapeutic agent is Taxol or Taxotere.

8. The method of claim 1 wherein said diphenyl compound is administered to the patient about 30 to about 90 minutes prior to said administration of said chemotherapeutic agents.

9. The method of claim 8 wherein said time is about 60 minutes.

10. The method of claim 7 wherein said diphenyl compound is administered by intravenous infusion of a solution thereof over a period of time of up to about 90 minutes prior to administration of said chemotherapeutic agents and is maintained during administration of said chemotherapeutic agents.

11. The method of claim 10 wherein said diphenyl compound is administered for about 60 minutes prior to administration of said chemotherapeutic agents and is maintained during intravenous infusion of said chemotherapeutic agents.

12. The method of claim 11 wherein administration of taxane chemotherapeutic agent, optionally in combination with anthracycline chemotherapeutic agent, is effected during about 20 minutes maintenance of infusion of diphenyl compound, followed by continued infusion of diphenyl compound for the remainder of the administration of the taxane chemotherapeutic agent.

13. The method of claim 8 wherein said diphenyl compound is administered in an amount of about 8 to about 240 mg/M² of said patient.

14. The method of claim 13 wherein said amount is about 3 to about 10 mg/kg of patient.

15. The method of claim 10 wherein said diphenyl compound is administered in an amount of about 3 to about 10 mg/kg of patient.

16. The method of claim 11 wherein said diphenyl compound is administered in an amount of about 6 mg/kg.

17. The method of claim 16 wherein said chemotherapeutic agents are administered in an amount of about 50 to about 60 mg/M² of patient for doxorubicin or epirubicin, about 175 to about 225 mg/M² for Taxol and about 75 to about 100 mg/M² of Taxotere.

18. The method of claim 16 wherein said anthracycline chemotherapeutic agent is doxorubicin or epirubicin administered in an amount of 50 mg/M² and said taxane therapeutic agent is Taxol administered in an amount of 175 mg/M² or Taxotere is administered in an amount of 75 mg/M².

19. The method of claim 1 wherein the number of cycles of chemotherapy treatment is about 5 to about 10 administered at intervals of about 21 to about 28 days.
